(19) United States
(12) Patent Application Publication
(10) Pub. No.: US 2017/0157173 A1
(43) Pub. Date: Jun. 8, 2017

(54) BIOCOMPATIBLE COLLOIDAL SOLUTION OF GOLD NANOPARTICLES IN NON-AQUEOUS POLAR SOLVENT AND METHOD OF OBTAINING THEREOF

(71) Applicant: TOVARISTVO Z OBMEZHENOU VIDPOVIDALNISTU "NANOMEDTRAST", Kyiv (UA)

(72) Inventors: Oleksandra Evgenivna RAIEVS'KA, Kyiv (UA); Oleksandr Leonidovych STROIUK, Kyiv (UA)

(73) Assignee: TOVARISTVO Z OBMEZHENOU VIDPOVIDALNISTU "NANOMEDTRAST", Kyiv (UA)

(21) Appl. No.: 15/324,034

(22) PCT Filed: Jul. 8, 2015
(86) PCT No.: PCT/UA2015/000057

§ 371 Date: Jan. 5, 2017

(30) Foreign Application Priority Data
Jul. 8, 2014 (UA) A2014 07693

(51) Int. Cl. A61K 33/24; B22F 9/24; A61K 8/04; A61K 9/10; A61K 8/60; A61K 9/16; A61K 8/67; A61K 8/34; A61K 8/22; B22F 1/00; A61K 8/19

(52) U.S. Cl. CPC A61K 33/24; B22F 1/0022; B22F 1/0074; B22F 9/24; A61K 8/04; A61K 9/10; A61K 8/19; A61K 9/1617; A61K 9/1611; A61K 9/1623; A61K 8/676; A61K 8/345; A61K 8/22; A61K 8/34; A61K 8/60; B22F 2009/245; B22F 2301/255; B22F 2304/054; B22F 2998/10

(57) ABSTRACT

The present application relates to colloidal chemistry, specifically to methods of synthesising gold nanoparticle colloids in a non-aqueous solvent, preferably, in dimethyl sulfoxide. In particular these gold nanoparticles have an average size of 5-20 nm and are in a biocompatible colloidal solution.

The invention relates to colloidal chemistry, specifically to methods of synthesising gold nanoparticle (NP) colloids in a non-aqueous solvent, and may be used, in particular, in the pharmaceutical or cosmetic industry, e.g. to produce gold nanoparticle colloids in a form suitable for introduction of gold and silver nanoparticles into soft dosage forms and cosmetic products—ointments, creams, etc.

The following colloidal solutions of gold nanoparticles, containing metallic gold in nanodispersed state stabilized with non-toxic products, are known to the applicant.

In particular, the prior art discloses a colloidal solution of gold nanoparticles and the method of obtaining thereof according to Ukrainian Patent No. 87744, published on Aug. 10, 2009, Publication No. 15, 2009, containing gold nanoparticles in an aqueous or aqueous-alcoholic solution comprising a carbohydrate, as a stabilizer, gold nanoparticles and water or an aqueous-alcoholic solution having components in the following ratios, mass %:

| | |
|---|---|
| gold nanoparticles | 0.000001-10. |
| carbohydrate | 1-90 |
| water or an aqueous-alcoholic solution | the rest. |

The prior art also discloses a colloidal solution of metal nanoparticles, in particular, gold or silver, and the method of obtaining thereof by reducing a metal salt in the presence of a chelating agent and a catalyst (international publication of application WO 2010/100107 dd. Sep. 10, 2010). A reductant was chosen from among glucose, galactose, maltose, lactose or sucrose. A chelating agent was chosen from among polyvinyl alcohol, polyvinylpyrrolidone, sodium lauryl sulphate, sodium dodecylbenzene sulphonate, cetyltrimethylammonium bromide, tetraoktylammonium bromide, Triton X-100, polyethylene glycol, ethylenediaminetetraacetic acid, starch, β-cyclodextrin β-CD. A catalyst was chosen from among hydroxides and carbonates of alkali metals, ammonia or urea. The resulting solution contained gold with the average particle size ranging from 20 to 30 nm for monodisperse distribution and from 5 to 10 nm for bimodal distribution.

The prior art also discloses the method of obtaining an aqueous suspension of precious metal colloid, in particular, gold, comprising reducing a precious metal salt in an aqueous solution using functionalized water-soluble quaternary ammonium salts in absence of organic solvents to form elementary nanoparticles (international publication of application WO/2009/096783 dd. Aug. 6, 2009). Functionalization of a soluble quaternary ammonium salt requires presence of at least one replacement group, such as CH₂OH or cyclohexenyl, preferably, in combination with at least one bulky group chosen from among C₆₊ alkyl, cycloalkyl, arylalkyl, or alkylaryl or aryl.

Further, the prior art discloses the method of obtaining a colloidal solution of precious metal nanoparticles, comprising dissolving gold iodide or silver iodide in water or a non-aqueous solvent, blowing gaseous carbon oxide (II) through the solution, followed by heating the solution up to 50° C., or adding an organic liquid, which does not mix with water or an non-aqueous solvent. Carbon tetrachloride in the amount up to 0.1 of the volume of the resulting solution, may be used as organic liquid (Patent RU 2357797 C2, published on Jun. 10, 2009).

The method of obtaining a colloidal solution containing extremely small particles of precious metals (Application JPS59145037 published on August, 1984) is also known in the prior art. According to the method, precious metal is reduced with a reducing agent in an aqueous solution of cyclodextrin previously dissolved by dissolving cyclodextrin (e.g., beta-cyclodextrin) in an aqueous solution of precious metal salt, such as rhodium (III) chloride, and the resulting solution is then treated with a reducing agent, such as, in particular, ethanol, to reduce precious metal. In this method, the molar ratio of cyclodextrin and metal salt is within 100-0.1. Furthermore, when ethanol is added to the aqueous solution of metal salt as a reducing agent, the resulting solution is heated at T=40-90° C. for the period from 3 minutes to several hours to obtain a colloidal solution of precious metal nanoparticles. The resulting colloidal solution contains precious metal nanoparticles with an average size of about 10-30 Å and, therefore, features extremely large surface area per unit of metal mass and high catalytic activity.

The prior art also discloses a colloidal solution of gold nanoparticles in a non-aqueous polar solvent, in particular, in dimethyl sulfoxide, and the method of obtaining thereof (Application JP2006205302 published Aug. 10, 2006), which is the closest among analogues. A solution of a non-aqueous polar solvent and an aqueous alkaline solution containing polysaccharides, in particular, a single-stranded complex with β-1,3-glucan (e.g. shizofilan), is mixed with a solution of metal nanoparticles. This provides conditions for introduction of metallic gold nanoparticles into hydrophobic inner space of a single-stranded complex.

The disadvantage of both the closest method and similar ones is obtaining aqueous colloidal solutions of precious metals in a form unsuitable for being introduced into soft dosage forms or cosmetic products—ointments and creams, since it is not combined with hydrophobic organic components of such forms. The majority of colloidal solutions of precious metals, known in the prior art, do not allow achieving the required biocompatibility level, so their use in the pharmaceutical and cosmetic industry is limited.

The invention is based on the object to produce a biocompatible colloidal solution of gold nanoparticles in a non-aqueous polar solvent in a state suitable for introduction of gold nanoparticles into soft dosage forms and cosmetic products—ointments and creams, and for combination of gold nanoparticles with hydrophobic organic components in ointments and creams by obtaining gold nanoparticles in those media of ointments and creams.

A further object of the invention was to produce a biocompatible colloidal solution of gold nanoparticles in a non-aqueous polar solvent free from oxalate anions, which are products of ascorbic acid conversion reaction, following ascorbic acid interaction with a gold salt and which can cause pain when ointments and creams, utilizing the claimed biocompatible colloidal solution of gold nanoparticles, is applied by using biocompatible reducing agents alternative to ascorbic acid.

This problem is solved, so that a biocompatible colloidal solution of gold nanoparticles in a non-aqueous polar solvent, preferably, in dimethyl sulfoxide comprises, according to the invention, gold nanoparticles obtained by reducing gold salt using a biocompatible reductant, which requires an alkaline medium to reduce gold ions to gold nanoparticles [Au⁰], and such alkaline medium is formed by tetraalkylammonium hydroxide, and ingredients are taken in amounts allowing to obtain nanoparticles with an average size of 5-20 nm, and the resulting colloid solution is adjusted to neutral pH.

A biocompatible reducing agent may comprise ascorbic acid or glycerine or hydrogen peroxide or ethyl alcohol or glucose.

Tetraalkylammonium hydroxide may comprise tetraethylammonium hydroxide or tetraisopropylammonium hydroxide or tetrabutylammonium hydroxide or tetrapentylammonium hydroxide.

Gold salt may comprise sodium tetrachloroaurate (III).

The average size of gold nanoparticles [Au⁰] may range 5 . . . 6 nm.

Further, the invention is based on the object to provide a method of obtaining a colloidal solution of gold nanoparticles in a non-aqueous polar solvent, suitable for introduction into soft dosage forms and cosmetic products and to interact with hydrophobic organic components in ointments and creams.

This problem is solved so that in the method of obtaining a colloidal solution of gold nanoparticles in a non-aqueous polar solvent, preferably, in dimethyl sulfoxide, a gold salt, according to the invention, is reduced with biocompatible reducing agent in an alkaline medium by interaction of gold salt solution and dimethyl sulfoxide solution with a biocompatible reductant solution, which requires an alkaline medium to reduce gold ions to gold nanoparticles [Au⁰], dimethyl sulfoxide and tetraalkylammonium hydroxide, subject to further adjustment of the resulting colloidal solution to neutral pH.

Neutral pH of the resulting colloidal solution may be adjusted by adding organic acid to the resulting colloidal solution.

Ascorbic acid or glycerine or hydrogen peroxide or ethyl alcohol or glucose may be used as a biocompatible reducing agent.

Tetraethylammonium hydroxide or tetraisopropylammonium hydroxide or tetrabutylammonium hydroxide or tetrapentylammonium hydroxide may be used as tetraalkylammonium hydroxide.

sodium tetrachloroaurate (III) may be used as a gold salt.

Further, optimal molar ratios were found empirically for components in the biocompatible colloidal solution of gold nanoparticles in a non-aqueous polar solvent, produced by the method disclosed herein, specifically:
molar ratio of the amount of gold in the form of nanoparticles to the amount of ascorbic acid and tetraalkylammonium hydroxide may be within 1:1:10,
molar ratio of the amount of gold in the form of nanoparticles to the amount of glycerine and tetraalkylammonium hydroxide may be within 1:1:10,
molar ratio of the amount of gold in the form of nanoparticles to the amount of hydrogen peroxide and tetraalkylammonium hydroxide may be within 1:15 . . . 100:10,
molar ratio of the amount of gold in the form of nanoparticles to the amount of ethyl alcohol and tetraalkylammonium hydroxide may be within 1:5 . . . 8:10,
molar ratio of the amount of gold in the form of nanoparticles to the amount of glucose and tetraalkylammonium hydroxide may be within 1:100 . . . 300:10.

The combination of essential features of the invention and the technical effect achieved, using the invention brings the following cause-and-effect result.

As it is known in the prior art, the most common method of obtaining highly stable gold colloids (sols) is Turkevich method (J. Kimling, M. Maier, B. Okenve, V. Kotaidis, H. Ballot, and A. Plech, Turkevich Method for Gold Nanoparticle Synthesis Revisited, Fachbereich Physik der Universität Konstanz, Universitätsstr. 10, D-78457 Konstanz, Germany, J. Phys. Chem. B, 2006, 110 (32), pp 15700-15707, DOI: 10.1021/jp061667w, Publication Date: Jul. 21, 2006). In this method, gold nanoparticles are obtained by chemical reduction of gold salt (NaAuCl₄) with citric or ascorbic acid (AA), which simultaneously act as a stabilizer of nanoparticles to prevent aggregation and consolidation. Interaction of gold salts with ascorbic acid is efficient in alkaline media only. Inorganic alkalis (NaOH, KOH), as conventionally used, are however insoluble in most dispersion media mentioned above.

To solve this problem, the inventors proposed to use an organic basis—tetraalkylammonium hydroxide, which is readily soluble in polar solvents mentioned above, in particular, in dimethyl sulfoxide (DMSO), a non-toxic, commonly used component of various warming pain-relieving ointments and creams, as alkali to reduce gold salt. To this end, the inventors obtained a biocompatible colloidal solution of gold nanoparticles using tetraethylammonium hydroxide or tetraisopropylammonium hydroxide or tetrabutylammonium hydroxide or tetrapentylammonium hydroxide as tetraalkylammonium hydroxide and confirmed that the solution claimed achieved the properties. The inventors, however, assume that obtaining the claimed biocompatible colloidal solution of gold nanoparticles is possible when other known alkyl groups are used in tetraalkylammonium hydroxide provided such groups are readily soluble in polar solvents.

The difference of the proposed method of obtaining a biocompatible colloidal solution of gold nanoparticles from Turkevich method is creating stronger alkaline medium by obtaining a mixture of two components: the first one being alkali based on sodium hydroxide or tetraethylammonium hydroxide etc. and the second one being sodium ascorbate or tetraethylammonium ascorbate (sodium ascorbate—as a mixture of AA and alkali). As a result, the conditions are now created for obtaining more concentrated solutions of gold nanoparticles. In Turkevich method described in the source above, concentration of gold [Au⁰] in the form of nanoparticles is 0.0005 mol/L, while the method claimed provides a concentration of [Au⁰] in the form of nanoparticles up to 0.002 mol/L and a narrower distribution by size, including, in particular, gold [Au⁰] nanoparticles with an average size within 5 . . . 6 nm. According to the method disclosed herein, after synthesis, the solution is neutralized by adding acetic or citric acid, and this does not change the system characteristics (except for pH value and ionic strength).

Further, while obtaining the claimed biocompatible colloidal solution of gold nanoparticles, the inventors used both ascorbic acid and biocompatible reducing agents alternative to ascorbic acid, such as, in particular, glycerine, hydrogen peroxide, ethyl alcohol and glucose. Notably, the list of these biocompatible reducing agents is not exhaustive, and other reducing agents may be known to those skilled in the art provided, however, the agents meet the single requirement to achieve the said technical effect—they reduce gold ions to metal in an alkaline medium. For example, there are a number of similar reducing agents, in particular, hydrazine, hydroquinone, formaldehyde, sodium borohydride, etc., however, the similar reducing agents do not meet the biocompatibility requirement and may not be used to obtain solutions of gold NPs to be used in the pharmaceutical or cosmetic industry.

The invention disclosed herein is illustrated by the following examples of obtaining a biocompatible colloidal solution of gold nanoparticles (NPs) using ascorbic acid (AA) as a reductant or using alternative reductants—glycerine, hydrogen peroxide, ethyl alcohol and glucose, and by the following graphic materials, specifically:

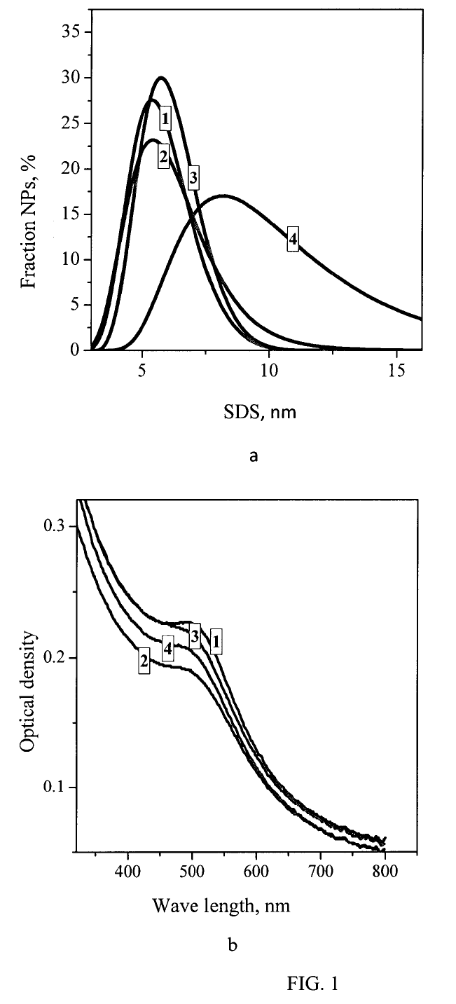

FIG. 1 shows distribution by solvodynamic size (a) and absorption spectra (b) of colloidal gold NPs synthetized with tetraethylammonium hydroxide (Et₄NOH, curves 1), tetrapropylammonium hydroxide (Pr₄NOH, curves 2), tetrabutylammonium hydroxide (Bu₄NOH, curves 3) and tetrapentylammonium hydroxide (Pt₄NOH, curves 4). [Au⁰]=2×10⁻³ mol/L, [AA]=1×10⁻³ mol/L, [OH⁻]=1×10⁻² mol/L. Cuvette−1.0 mm (for this and subsequent distributions);

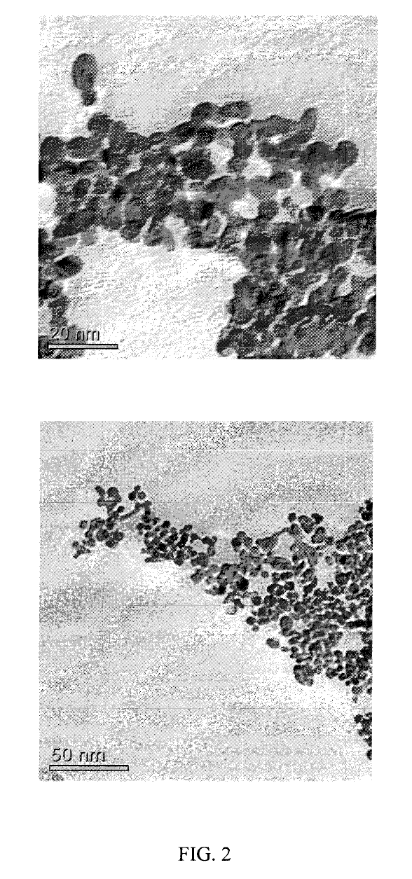

FIG. 2 shows electron microphotographs of colloidal gold NPs synthetized in Example 1;

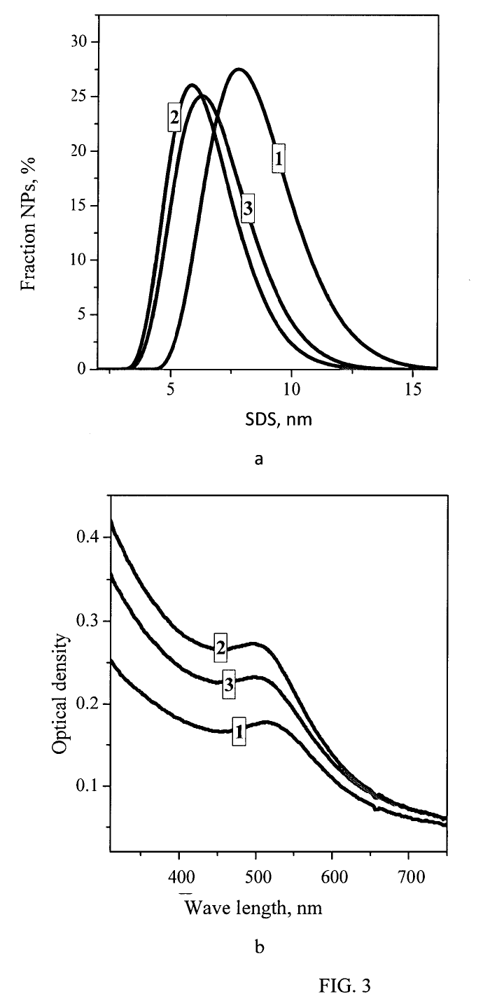

FIG. 3 shows distribution by solvodynamic size (a) and absorption spectra (b) of colloidal gold NPs synthetized with various amounts of Et₄NOH: 5×10⁻³ mol/L (curves 1), 1×10⁻² mol/L (curves 2), 2×10⁻² mol/L (curves 3). [Au⁰]=2×10⁻³ mol/L, [AA]=1×10⁻³ mol/L, [OH⁻]=1×10⁻² mol/L;

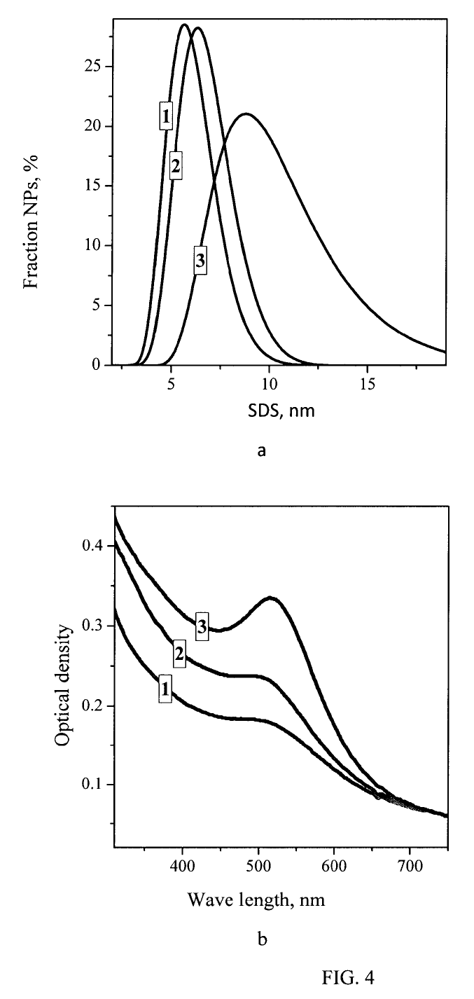

FIG. 4 shows distribution by solvodynamic size (a) and absorption spectra (b) of colloidal gold NPs synthetized with 1×10⁻² mol/L Et₄NOH and various amounts of AA: 5×10⁻⁴ mol/L (curves 1), 1×10⁻³ mol/L (curves 2), 2×10⁻³ mol/L (curves 3). [Au⁰]=1×10⁻³ mol/L;

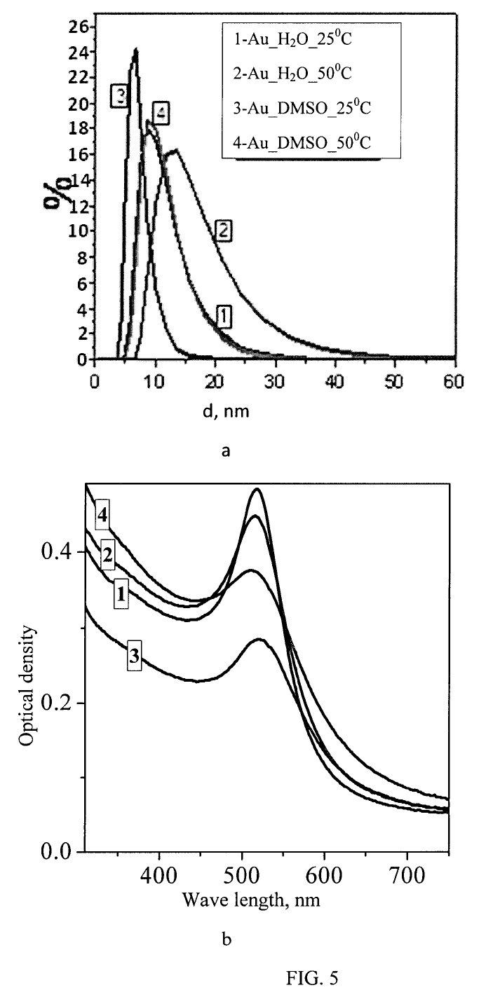

FIG. 5 shows distribution by solvodynamic size (a) and absorption spectra (b) of colloidal gold NPs synthetized in water (curves 1 and 2) and DMSO (curves 3 and 4) at 25° C. (curves 1 and 3) and 50° C. (curves 2 and 4). Synthesis in water: [Au⁰]=1×10⁻³ mol/L, [AA]=1×10⁻³ mol/L, [NaOH]=1×10⁻² mol/L. Synthesis in DMSO: [Au⁰]=1×10⁻³ mol/L, [AA]=1×10⁻³ mol/L, [Et₄NOH]=1×10⁻² mol/L;

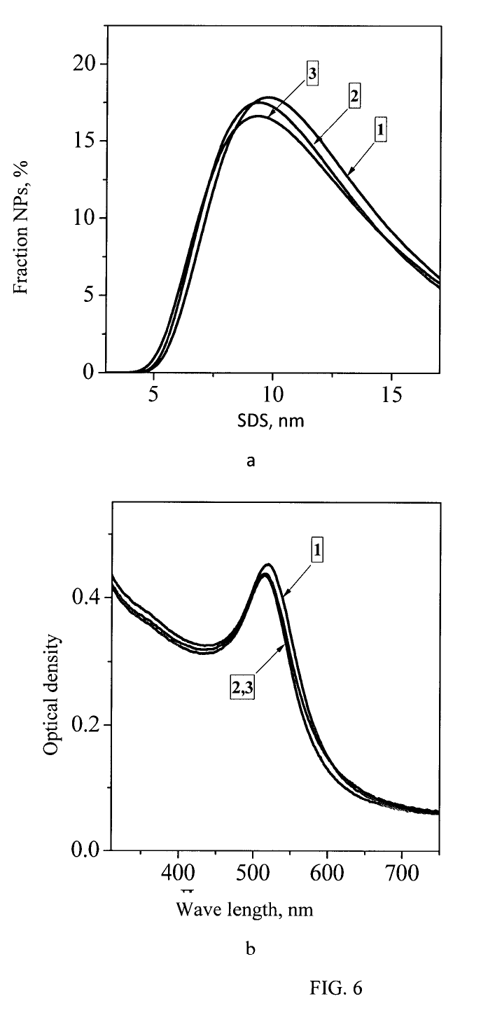

FIG. 6 shows distribution by solvodynamic size (a) and absorption spectra (b) of colloidal gold NPs, synthetized in water at 25° C. in the absence of post-synthesis additives (curves 1) and after adding of 8×10⁻³ mol/L of acetic acid (curves 2) or 2.7 10⁻³ M of citric acid (curves 3) at the post-synthesis stage. [Au⁰] 1×10⁻³ mol/L, [AA]=1×10⁻³ mol/L, [NaOH]=1×10⁻² mol/L;

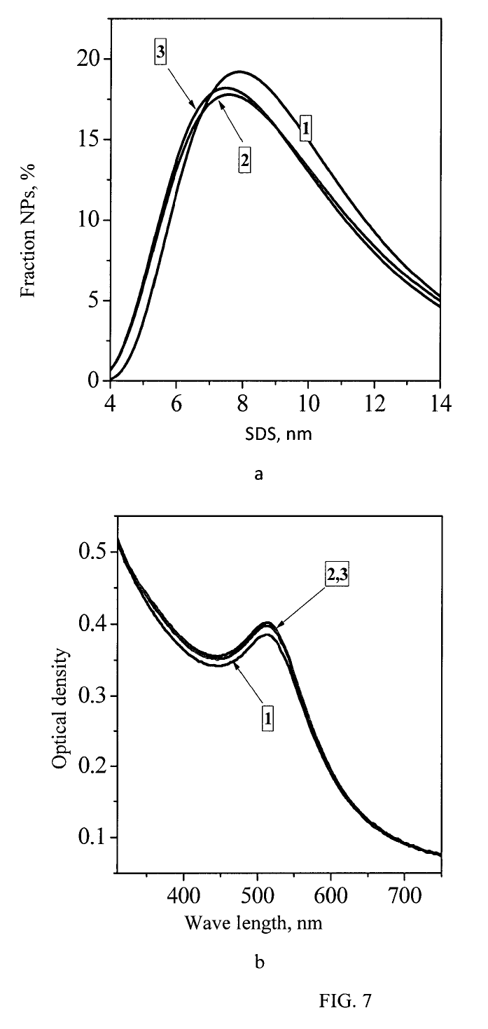

FIG. 7 shows distribution by solvodynamic size (a) and absorption spectra (b) of gold NPs, synthetized in DMSO at 25° C. in the absence of post-synthesis additives (curves 1) and after adding 8×10⁻³ mol/L of acetic acid (curves 2) or 2.7 10⁻³ M of citric acid (curves 3) at the post-synthesis stage. [Au⁰]=1×10⁻³ mol/L, [AA]=1×10⁻³ mol/L, [Et₄NOH]=1×10⁻² mol/L;

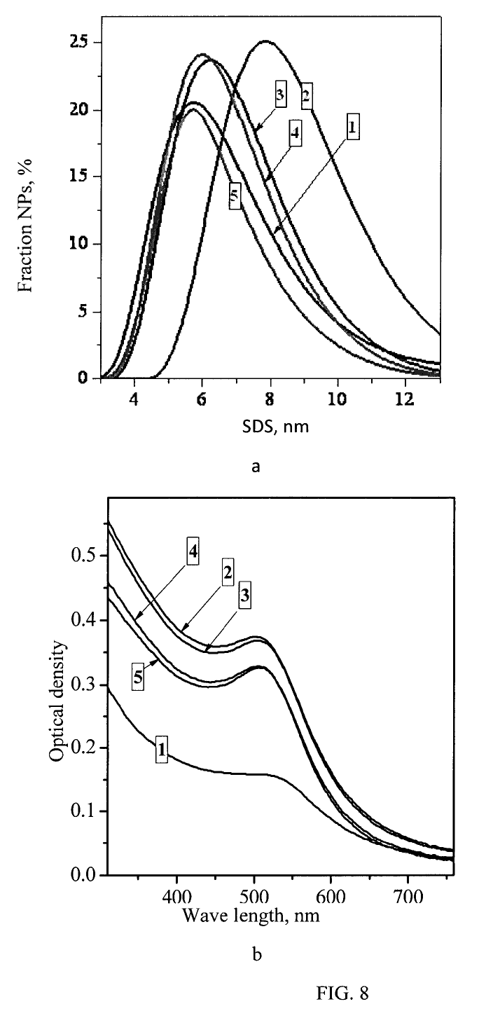

FIG. 8 shows distribution by solvodynamic size (a) and absorption spectra (b) for gold NPs, synthetized in DMSO at 25° C., using various quantities of glycerine: 0.1% (curves 1), 0.15% (curves 2), 0.25% (curves 3), 0.35% (curves 4) and 0.50% (curves 5). [Au⁰]=1×10⁻³ mol/L, [Et₄NOH]=1×10⁻² mol/L;

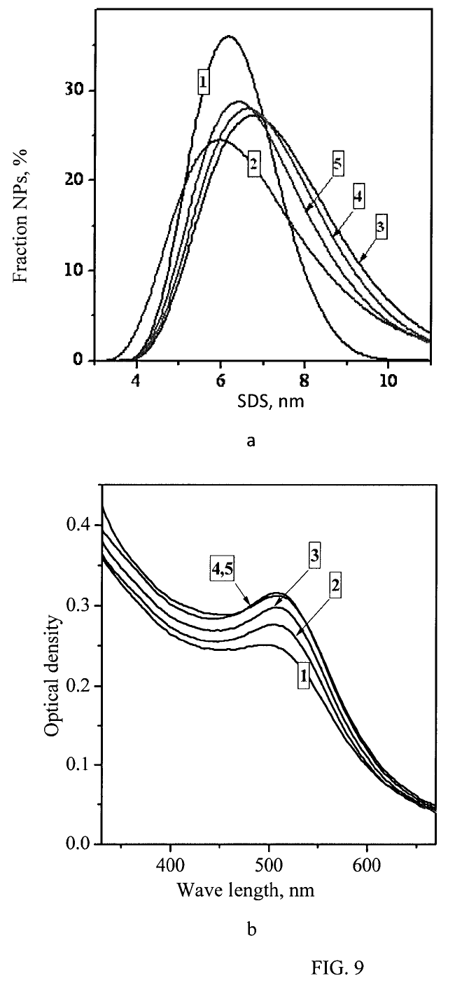

FIG. 9 shows distribution by solvodynamic size (a) and absorption spectra (b) for gold NPs, synthetized in DMSO at 25° C., using various quantities of glucose: 0.1% (curves 1), 0.2% (curves 2), 0.3% (curves 3), 0.4% (curves 4) and 0.50% (curves 5). [Au⁰]=1×10⁻³ mol/L, [Et₄NOH]=1×10⁻² mol/L;

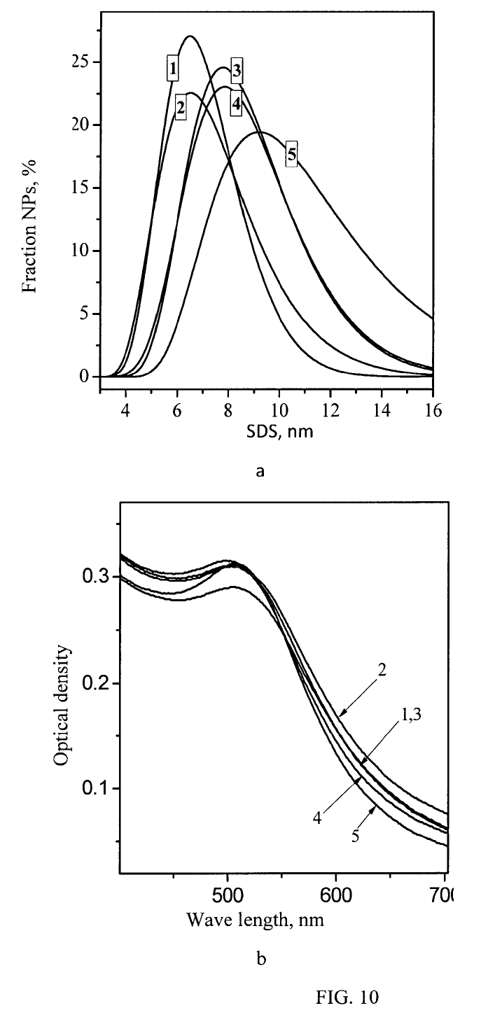

FIG. 10 shows distribution by solvodynamic size (a) and absorption spectra (b) for gold NPs, synthetized in DMSO at 25° C., using various quantities of ethyl alcohol: 0.5% (curves 1), 0.75% (curves 2), 1.0% (curves 3), 1.5% (curves 4) and 2.0% (curves 5). [Au⁰]=1×10⁻³ mol/L, [Et₄NOH]=1×10⁻² mol/L;

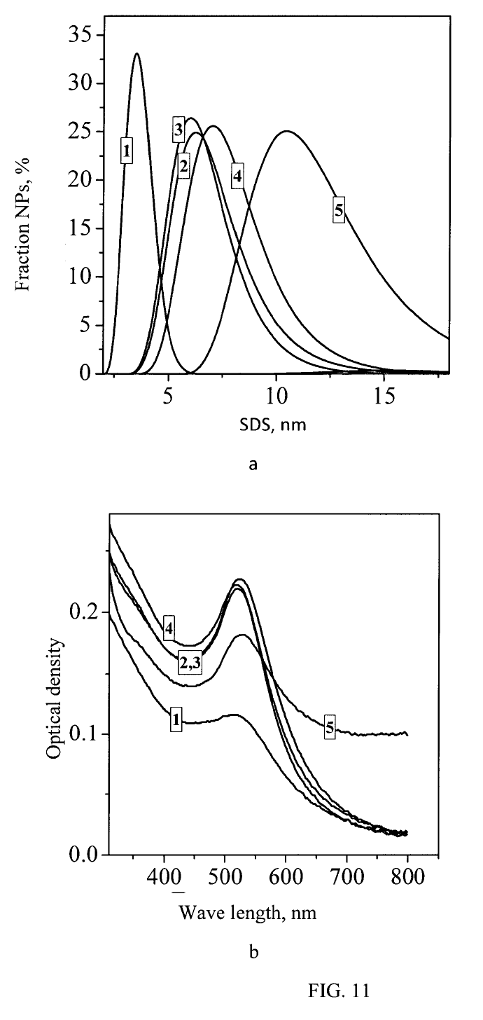

FIG. 11 shows distribution by solvodynamic size (a) and absorption spectra (b) for gold NPs, synthetized in DMSO at 25° C., using various quantities of hydrogen peroxide (H₂O₂): 0.01% (curves 1), 0.02% (curves 2), 0.03% (curves 3), 0.04% (curves 4) and 0.05% (curves 5). [Au⁰]=1×10⁻³ mol/L, [Et₄NOH]=1×10⁻² mol/L;

Graphic materials, which illustrate the invention disclosed herein, and an example of the resulting biocompatible colloidal solution of gold nanoparticles and the method obtaining thereof are not intended to restrict the scope of claims thereto, but explain the essence of the invention only.

In the first panel of examples, a biocompatible colloidal solution of gold nanoparticles (NPs) was obtained using ascorbic acid (AA) as a reductant and tetraalkylammonium, hydroxides having various alkyl groups, to form an alkaline medium.

### EXAMPLE NO. 1

Obtaining a biocompatible colloidal solution of gold NPs, using hydroxide tetraethylammonium. Two separate solutions were prepared first. For the first solution, 0.1 ml of 1.0 mol/L aqueous NaAuCl₄ solution was added to 4.7 mL of DMSO under stirring. For the second solution, 5 mL of DMSO, 0.1 mL of 1 mol/L of aqueous hydroxide tetraethylammonium (Et₄NOH) solution and 0.1 mL of 0.1 mol/L aqueous AA solution were mixed. Both solutions were further mixed under vigorous stirring. This formed a solution of gold NPs, containing [Au⁰]=1×10⁻³ mol/L. Distribution of gold NPs by solvodynamic size (SDS) and colloid absorption spectrum are demonstrated by curves 1 on FIG. 1a and FIG. 1b, respectively.

For this and subsequent panels of examples, a standard magnetic mixer, 300 rpm, was used for stirring. NPs are synthetized at a room temperature in the air.

### EXAMPLE NO. 2

Obtaining a biocompatible colloidal solution of gold NPs using hydroxide tetraisopropylammonium. Two separate solutions were prepared first. For the first solution, 0.1 ml of 1.0 mol/L aqueous NaAuCl₄ solution was added to 4.7 mL of DMSO under stirring. For the second solution, 5 mL of DMSO, 0.1 mL of 1 mol/L of aqueous hydroxide tetraisopropylammonium (Pr₄NOH) solution and 0.1 mL of 0.1 mol/L aqueous AA solution were mixed. Both solutions were further mixed under vigorous stirring. This formed a solution of gold NPs containing [Au⁰]=1×10⁻³ mol/L. Distribution of gold NPs by SDS and colloid absorption spectrum are demonstrated by curves 2 on FIG. 1a and FIG. 1b, respectively.

### EXAMPLE NO. 3

Obtaining a biocompatible colloidal solution of gold NPs using tetrabutylammonium hydroxide. Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous tetrabutylammonium hydroxide (Bt₄NOH) solution and 0.1 mL of 0.1 mol/L aqueous AA solution were mixed. Both solutions were further mixed under vigorous stirring. This formed a solution of gold NPs containing [Au⁰]=1×10⁻³ mol/L. Distribution of gold NPs by SDS and colloid absorption spectrum are demonstrated by curves 3 on FIG. 1a and FIG. 1b, respectively.

### EXAMPLE NO. 4

Obtaining a biocompatible colloidal solution of gold NPs using tetrapentylammonium hydroxide. Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous tetrapentylammonium hydroxide (Pt₄NOH) solution and 0.1 mL of 0.1 mol/L aqueous AA solution were mixed. Both solutions were further mixed under vigorous stirring. This formed a solution of gold NPs containing [Au⁰]=1×10⁻³ mol/L. Distribution of gold NPs by SDS and colloid absorption spectrum are demonstrated by curves 4 on FIG. 1a and FIG. 1b, respectively.

As shown in FIG. 1, distribution of gold NPs, obtained using various tetraalkylammonium hydroxides, and absorption spectra thereof are almost identical. Therefore, the subsequent syntheses were carried out, using the cheapest tetraalkylammonium hydroxide. FIG. 2 shows gold NPs, obtained with this method. The data shows, that the average size of NPs is 5-6 nm, that is consistent with findings of dynamic light scattering spectroscopy.

The second panel of examples was intended to select optimal concentration of tetraethylammonium hydroxide to obtain gold NPs, having maximum stability and minimum SDS.

### EXAMPLE NO. 5

Two separate solutions were prepared first. For the first solution, 0.1 ml of 0.1 mol/L aqueous NaAuCl₄ solution was added to 4.75 mL of DMSO under stirring. For the second solution, 5 mL of DMSO, 0.05 mL of 1 mol/L of aqueous Et₄NOH solution and 0.1 mL of 0.1 mol/L aqueous AA solution were mixed. Both solutions were further mixed under vigorous stirring. This formed a solution of gold NPs, containing [Au⁰]=1×10⁻³ mol/L. Distribution of gold NPs by SDS and colloid absorption spectrum are demonstrated by curves 1 on FIG. 3a and FIG. 3b, respectively.

### EXAMPLE NO. 6

Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous Et₄NOH solution and 0.1 mL of 0.1 mol/L aqueous AA solution were mixed. Both solutions were further mixed under vigorous stirring. This formed a solution of gold NPs, containing [Au⁰]=1×10⁻³ mol/L. Distribution of gold NPs by SDS and colloid absorption spectrum are demonstrated by curves 2 on FIG. 3a and FIG. 3b, respectively.

### EXAMPLE NO. 7

Two separate solutions were prepared first. For the first solution, 0.1 ml of 0.1 mol/L aqueous NaAuCl₄ solution was added to 4.6 mL of DMSO under stirring. For the second solution, 5 mL of DMSO, 0.2 mL of 1 mol/L of aqueous Et₄NOH solution and 0.1 mL of 0.1 mol/L aqueous AA solution were mixed. Both solutions were further mixed under vigorous stirring. This formed a solution of gold NPs, containing [Au⁰]=1×10⁻³ mol/L. Distribution of gold NPs by SDS and colloid absorption spectrum are demonstrated by curves 3 on FIG. 3a and FIG. 3b, respectively.

Conclusion: As demonstrated by the examples and SDS distribution (a) and absorption spectra (b) of colloid gold NPs, shown in FIG. 3, stable colloidal solutions of gold NPs with the smallest SDS are produced when 1×10⁻² mol/L tetraethylammonium hydroxide is used.

In the third panel of examples, the optimal concentration of reducing agent, ascorbic acid, was selected to produce gold NPs having maximum stability and minimum SDS.

### EXAMPLE NO. 8

Two separate solutions were prepared first. For the first solution, 0.1 ml of 0.1 mol/L aqueous NaAuCl₄ solution was added to 4.75 mL of DMSO under stirring. For the second solution, 5 mL of DMSO, 0.1 mL of 1 mol/L of aqueous Et₄NOH solution and 0.05 mL of 0.1 mol/L aqueous AA solution were mixed. Both solutions were further mixed under vigorous stirring. This formed a solution of gold NPs, containing [Au⁰]=1×10⁻³ mol/L. Distribution of gold NPs by SDS and colloid absorption spectrum are demonstrated by curves 1 on FIG. 4a and FIG. 4b, respectively.

### EXAMPLE NO. 9

Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous Et₄NOH solution and 0.1 mL of 0.1 mol/L aqueous AA solution were mixed. Both solutions were further mixed under vigorous stirring. This formed a solution of gold NPs, containing [Au⁰]=1×10⁻³ mol/L. Distribution of gold NPs by SDS and colloid absorption spectrum are demonstrated by curves 2 on FIG. 4a and FIG. 4b, respectively.

### EXAMPLE NO. 10

Two separate solutions were prepared first. For the first solution, 0.1 ml of 0.1 mol/L aqueous NaAuCl₄ solution was added to 4.6 mL of DMSO under stirring. For the second solution, 5 mL of DMSO, 0.1 mL of 1 mol/L of aqueous Et₄NOH solution and 0.2 mL of 0.1 mol/L aqueous AA solution were mixed. Both solutions were further mixed under vigorous stirring. This formed a solution of gold NPs containing [Au⁰]=1×10⁻³ mol/L. Distribution of gold NPs by SDS and colloid absorption spectrum are demonstrated by curves 3 on FIG. 4a and FIG. 4b, respectively.

Conclusion: As demonstrated by the examples and SDS distribution (a) and absorption spectra (b) of colloid gold NPs, stable colloidal solutions of gold NPs with the smallest SDS may be produced when 1×10⁻³ mol/L ascorbic acid is used.

The fourth panel of examples studied whether it is possible to obtain aqueous colloids of gold NPs, using the method disclosed herein, and what is the impact of the nature of the solvent and synthesis temperature on SDS and spectral characteristics of gold NPs.

### EXAMPLE NO. 11

Two separate solutions were prepared first. For the first solution, 0.1 ml of 0.1 mol/L aqueous NaAuCl₄ solution was added to 4.7 mL of water under stirring. For the second solution, 5 mL of water, 0.1 mL of 1 mol/L of aqueous sodium hydroxide solution and 0.1 mL of 0.1 mol/L aqueous AA solution were mixed. Both solutions were further mixed under vigorous stirring at 25° C. This formed a solution of gold NPs, containing [Au⁰]=1×10⁻³ mol/L. Distribution of gold NPs by SDS and colloid absorption spectrum are demonstrated by curves 1 on FIG. 5a and FIG. 5b, respectively.

### EXAMPLE NO. 12

Two separate solutions were prepared first. For the first solution, 0.1 ml of 0.1 mol/L aqueous NaAuCl₄ solution was added at 50° C. to 4.7 mL of water under stirring. For the second solution, 5 mL of water, 0.1 mL of 1 mol/L of aqueous sodium hydroxide solution and 0.1 mL of 0.1 mol/L aqueous AA solution were mixed also at 50° C. Both solutions were further mixed under vigorous stirring at 50° C. This formed a solution of gold NPs, containing [Au⁰]=1×10⁻³ mol/L. Distribution of gold NPs by SDS and colloid absorption spectrum are demonstrated by curves 3 on FIG. 5a and FIG. 5b, respectively.

### EXAMPLE NO. 13

Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous Et₄NOH solution and 0.1 mL of 0.1 mol/L aqueous AA solution were mixed. Both solutions were further mixed under vigorous stirring at 25° C. This formed a solution of gold NPs containing [Au⁰]=1×10⁻³ mol/L. Distribution of gold NPs by SDS and colloid absorption spectrum are demonstrated by curves 3 on FIG. 5a and FIG. 5b, respectively.

### EXAMPLE NO. 14

Two separate solutions were prepared first. For the first solution, 0.1 ml of 0.1 mol/L aqueous NaAuCl₄ solution was added at 50° C. to 4.7 mL of DMSO under stirring. For the second solution, 5 mL of DMSO, 0.1 mL of 1 mol/L of aqueous Et₄NOH solution and 0.1 mL of 0.1 mol/L aqueous AA solution were mixed also at 50° C. Both solutions were further mixed under vigorous stirring at T=50° C. This formed a solution of gold NPs containing [Au⁰]=1×10⁻³ mol/L. Distribution of gold NPs by SDS and colloid absorption spectrum are demonstrated by curves 4 on FIG. 5a and FIG. 5b, respectively.

Conclusion: As demonstrated by the examples and SDS distribution (a) and absorption spectra (b) of gold colloids, gold NPs, produced in DMSO and water in the presence of organic hydroxides, described above, have almost identical characteristics. Furthermore, synthesis temperature and post-synthesis treatment at 25-50° C. practically do not affect the aggregation stability and average solvodynamic size of particles.

For further use in pharmacology, colloids, so obtained, should have neutral pH. The fifth panel of experiments studied the impact of neutralisation of residual alkali with citric or acetic acid in a colloid on its SDS and spectral profiles. Baseline gold colloids correspond to Examples No. 11 (water) and 13 (DMSO). Distribution of baseline gold NPs by SDS and colloid absorption spectrum are demonstrated by curves 1 on FIG. 6a and FIG. 6b (for water) and FIG. 7a and FIG. 7b (for DMSO).

### EXAMPLE NO. 15

Two separate solutions were prepared first. For the first solution, 0.1 ml of 0.1 mol/L aqueous NaAuCl₄ solution was added to 4.7 mL of DMSO under stirring. For the second solution, 5 mL of water, 0.1 mL of 1 mol/L of aqueous sodium hydroxide solution and 0.1 mL of 0.1 mol/L aqueous AA solution were mixed. Both solutions were further mixed under vigorous stirring. The concentration of gold in the colloid is [Au⁰]=1×10⁻³ mol/L. 0.8 mL of 0.1 mol/L of aqueous acetic acid solution was added to the colloid. Distribution of gold NPs by SDS and colloid absorption spectrum are demonstrated by curves 2 on FIG. 6a and FIG. 6b, respectively.

### EXAMPLE NO. 16

Two separate solutions were prepared first. For the first solution, 0.1 ml of 0.1 mol/L aqueous NaAuCl₄ solution was added to 4.7 mL of water under stirring. For the second solution, 5 mL of water, 0.1 mL of 1 mol/L of aqueous sodium hydroxide solution and 0.1 mL of 0.1 mol/L aqueous AA solution were mixed. Both solutions were further mixed under vigorous stirring. The concentration of gold in the colloid is [Au⁰]=1×10⁻³ mol/L. 0.27 mL of 0.1 mol/L of aqueous citric acid solution was added to the colloid. Distribution of gold NPs by SDS and colloid absorption spectrum are demonstrated by curves 3 on FIG. 5a and FIG. 5b, respectively.

### EXAMPLE NO. 17

Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous Et₄NOH solution and 0.1 mL of 0.1 mol/L aqueous AA solution were mixed. Both solutions were further mixed under vigorous stirring. The concentration of gold in the colloid is [Au⁰]=1×10⁻³ mol/L. 0.8 mL of 0.1 mol/L of aqueous acetic acid solution was added to the colloid. Distribution of gold NPs by SDS and colloid absorption spectrum are demonstrated by curves 2 on FIG. 7a and FIG. 7b, respectively.

### EXAMPLE NO. 18

Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous Et₄NOH solution and 0.1 mL of 0.1 mol/L aqueous AA solution were mixed. Both solutions were further mixed under vigorous stirring. The concentration of gold in the colloid is [Au⁰]=1×10⁻³ mol/L. 0.27 mL of 0.1 mol/L of aqueous citric acid solution was added to the colloid. Distribution of gold NPs by SDS and colloid absorption spectrum are demonstrated by curves 3 on FIG. 7a and FIG. 7b, respectively.

Conclusion: Neutralisation of residual alkali in colloids in water and DMSO by adding citric or acetic acid at a post-synthesis stage do not practically change characteristics and stability of gold NPs.

A further panel of experiments was intended to identify opportunities of obtaining gold NPs in DMSO using other biocompatible reducing agents, alternatives to ascorbic acid, in particular, such as glycerine, glucose, hydrogen peroxide and ethanol. Alternative biocompatible reducing agents may be used to relieve short-term pain syndrome associated with the presence of oxalate anion, a product of ascorbic acid oxidation, following intramuscular or intravenous administration of such medicinal product.

A further panel of experiments confirmed the possibility of using glycerine as a reducing agent in the method disclosed herein to produce a biocompatible colloidal solution of gold nanoparticles in a non-aqueous polar solvent.

### EXAMPLE NO. 19

Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous Et₄NOH solution and 0.1 mL of 10% aqueous glycerine solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 1 on FIG. 8a and FIG. 8b, respectively.

### EXAMPLE NO. 20

Two separate solutions were prepared first. For the first solution, 0.1 ml of 0.1 mol/L aqueous NaAuCl₄ solution was added to 4.65 mL of DMSO under stirring. For the second solution, 5 mL of DMSO, 0.1 mL of 1 mol/L of aqueous Et₄NOH solution and 0.15 mL of 10% aqueous glycerine solution were mixed. Both solutions were further mixed under vigorous stirring. This forms a solution of gold NPs containing [Au⁰]=1×10⁻³ mol/L. Distribution of gold NPs by SDS and colloid absorption spectrum are demonstrated by curves 2 on FIG. 8a and FIG. 8b, respectively.

### EXAMPLE NO. 21

Two separate solutions were prepared first. For the first solution, 0.1 ml of 0.1 mol/L aqueous NaAuCl₄ solution was added to 4.55 mL of DMSO under stirring. For the second solution, 5 mL of DMSO, 0.1 mL of 1 mol/L of aqueous Et₄NOH solution and 0.25 mL of 10% aqueous glycerine solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 3 on FIG. 8a and FIG. 8b, respectively.

### EXAMPLE NO. 22

Two separate solutions were prepared first. For the first solution, 0.1 ml of 0.1 mol/L aqueous NaAuCl₄ solution was added to 4.45 mL of DMSO under stirring. For the second solution, 5 mL of DMSO, 0.1 mL of 1 mol/L of aqueous Et₄NOH solution and 0.35 mL of 10% aqueous glycerine solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 4 on FIG. 8a and FIG. 8b, respectively.

### EXAMPLE NO. 23

Two separate solutions were prepared first. For the first solution, 0.1 ml of 0.1 mol/L aqueous NaAuCl₄ solution was added to 4.3 mL of DMSO under stirring. For the second solution, 5 mL of DMSO, 0.1 mL of 1 mol/L of aqueous Et₄NOH solution and 0.5 mL of 10% aqueous glycerine solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 5 on FIG. 8a and FIG. 8b, respectively.

The next panel of experiments confirmed the possibility to use glucose as a reducing agent in the method disclosed herein.

### EXAMPLE NO. 24

Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous Et₄NOH solution and 0.1 mL of 10% aqueous glucose solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 1 on FIG. 9a and FIG. 9b, respectively.

### EXAMPLE NO. 25

Two separate solutions were prepared first. For the first solution, 0.1 ml of 0.1 mol/L aqueous NaAuCl₄ solution was added to 4.6 mL of DMSO under stirring. For the second solution, 5 mL of DMSO, 0.1 mL of 1 mol/L of aqueous Et₄NOH solution and 0.2 mL of 10% aqueous glucose solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 2 on FIG. 9a and FIG. 9b, respectively.

### EXAMPLE NO. 26

Two separate solutions were prepared first. For the first solution, 0.1 ml of 0.1 mol/L aqueous NaAuCl₄ solution was added to 4.5 mL of DMSO under stirring. For the second solution, 5 mL of DMSO, 0.1 mL of 1 mol/L of aqueous Et₄NOH solution and 0.3 mL of 10% aqueous glucose solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 3 on FIG. 9a and FIG. 9b, respectively.

### EXAMPLE NO. 27

Two separate solutions were prepared first. For the first solution, 0.1 ml of 0.1 mol/L aqueous NaAuCl₄ solution was added to 4.4 mL of DMSO under stirring. For the second solution, 5 mL of DMSO, 0.1 mL of 1 mol/L of aqueous Et₄NOH solution and 0.4 mL of 10% aqueous glucose solution were mixed. Both solutions were further mixed under vigorous stirring. This foul's a solution of gold NPs, containing [Au⁰]=1×10⁻³ mol/L. Distribution of gold NPs by SDS and colloid absorption spectrum are demonstrated by curves 4 on FIG. 9a and FIG. 9b, respectively.

### EXAMPLE NO. 28

Two separate solutions were prepared first. For the first solution, 0.1 ml of 0.1 mol/L aqueous NaAuCl₄ solution was added to 4.3 mL of DMSO under stirring. For the second solution, 5 mL of DMSO, 0.1 mL of 1 mol/L of aqueous Et₄NOH solution and 0.5 mL of 10% aqueous glucose solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 5 on FIG. 9a and FIG. 9b, respectively.

The next panel of experiments confirmed the possibility to use ethyl alcohol as a reducing agent in the method disclosed herein.

### EXAMPLE NO. 29

Two separate solutions were prepared first. For the first solution, 0.1 ml of 0.1 mol/L aqueous NaAuCl₄ solution was added to 4.75 mL of DMSO under stirring. For the second solution, 5 mL of DMSO, 0.1 mL of 1 mol/L of aqueous Et₄NOH solution and 0.05 mL of 10% aqueous ethyl alcohol solution were mixed. Both solutions were further mixed under vigorous stirring. This forms a solution of gold NPs containing [Au⁰]=1×10⁻³ mol/L. Distribution of gold NPs by SDS and colloid absorption spectrum are demonstrated by curves 1 on FIG. 10a and FIG. 10b, respectively.

### EXAMPLE NO. 30

Two separate solutions were prepared first. For the first solution, 0.1 ml of 0.1 mol/L aqueous NaAuCl₄ solution was added to 4.725 mL of DMSO under stirring. For the second solution, 5 mL of DMSO, 0.1 mL of 1 mol/L of aqueous Et₄NOH solution and 0.075 mL of 10% aqueous ethyl alcohol solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 2 on FIG. 10a and FIG. 10b, respectively.

### EXAMPLE NO. 31

Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous Et₄NOH solution and 0.1 mL of 10% aqueous ethyl alcohol solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 3 on FIG. 10a and FIG. 10b, respectively.

### EXAMPLE NO. 32

Two separate solutions were prepared first. For the first solution, 0.1 ml of 0.1 mol/L aqueous NaAuCl₄ solution was added to 4.65 mL of DMSO under stirring. For the second solution, 5 mL of DMSO, 0.1 mL of 1 mol/L of aqueous Et₄NOH solution and 0.15 mL of 10% aqueous ethyl alcohol solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 4 on FIG. 10a and FIG. 10b, respectively.

### EXAMPLE NO. 33

Two separate solutions were prepared first. For the first solution, 0.1 ml of 0.1 mol/L aqueous NaAuCl₄ solution was added to 4.6 mL of DMSO under stirring. For the second solution, 5 mL of DMSO, 0.1 mL of 1 mol/L of aqueous Et₄NOH solution and 0.2 mL of 10% aqueous ethyl alcohol solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 5 on FIG. 10a and FIG. 10b, respectively.

The next panel of experiments confirmed the possibility to use hydrogen peroxide as a reducing agent in the method disclosed herein.

### EXAMPLE NO. 34

Two separate solutions were prepared first. For the first solution, 0.1 ml of 0.1 mol/L aqueous NaAuCl₄ solution was added to 4.7 mL of DMSO under stirring. For the second solution, 5 mL of DMSO, 0.1 mL of 1 mol/L of aqueous Et₄NOH solution and 0.1 mL of 1% aqueous hydrogen peroxide solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 1 on FIG. 11a and FIG. 11b, respectively.

### EXAMPLE NO. 35

Two separate solutions were prepared first. For the first solution, 0.1 ml of 0.1 mol/L aqueous NaAuCl₄ solution was added to 4.6 mL of DMSO under stirring. For the second solution, 5 mL of DMSO, 0.1 mL of 1 mol/L of aqueous Et₄NOH solution and 0.2 mL of 1% aqueous hydrogen peroxide solution were mixed. Both solutions were further mixed under vigorous stirring. This forms a solution of gold NPs containing [Au⁰]=1×10⁻³ mol/L. Distribution of gold NPs by SDS and colloid absorption spectrum are demonstrated by curves 2 on FIG. 11a and FIG. 11b, respectively.

### EXAMPLE NO. 36

Two separate solutions were prepared first. For the first solution, 0.1 ml of 0.1 mol/L aqueous NaAuCl₄ solution was added to 4.5 mL of DMSO under stirring. For the second solution, 5 mL of DMSO, 0.1 mL of 1 mol/L of aqueous Et₄NOH solution and 0.3 mL of 1% aqueous hydrogen peroxide solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 3 on FIG. 11a and FIG. 11b, respectively.

### EXAMPLE NO. 35

Two separate solutions were prepared first. For the first solution, 0.1 ml of 0.1 mol/L aqueous NaAuCl₄ solution was added to 4.4 mL of DMSO under stirring. For the second solution, 5 mL of DMSO, 0.1 mL of 1 mol/L of aqueous Et₄NOH solution and 0.4 mL of 1% aqueous hydrogen peroxide solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 4 on FIG. 11a and FIG. 11b, respectively.

### EXAMPLE NO. 36

Two separate solutions were prepared first. For the first solution, 0.1 ml of 0.1 mol/L aqueous NaAuCl₄ solution was added to 4.3 mL of DMSO under stirring. For the second solution, 5 mL of DMSO, 0.1 mL of 1 mol/L of aqueous Et₄NOH solution and 0.5 mL of 1% aqueous hydrogen peroxide solution were mixed. Both solutions were further mixed under vigorous stirring. This forms a solution of gold NPs, containing [Au⁰]=1×10⁻³ mol/L. Distribution of gold NPs by SDS and colloid absorption spectrum are demonstrated by curves 5 on FIG. 11a and FIG. 11b, respectively.

Therefore, the invention disclosed herein allows obtaining a biocompatible colloidal solution of gold nanoparticles in a non-aqueous polar solvent in a form suitable for introduction of gold nanoparticles into soft dosage forms and cosmetic products—ointments and creams, and obtaining a biocompatible colloidal solution of gold nanoparticles in a non-aqueous polar solvent, the use of which avoids pain associated with administration of the product.

1. A biocompatible colloidal solution of gold nanoparticles in a non-aqueous polar solvent, preferably, in dimethyl sulfoxide, characterized in that the solution contains gold nanoparticles, obtained by reducing a gold salt, using a biocompatible reductant, which requires an alkaline medium to reduce gold ions to gold nanoparticles [Au⁰], and the alkaline medium is obtained with tetraalkylammonium hydroxide, and the ingredients are taken in such amount that allows obtaining nanoparticles with an average size of 5-20 nm, and the resulting colloidal solution is adjusted to neutral pH.

2. The colloid solution of claim 1, wherein ascorbic agent is a biocompatible reducing agent.

3. The colloid solution of claim 1, wherein glycerine is a biocompatible reducing agent.

4. The colloid solution of claim 1, wherein hydrogen peroxide is a biocompatible reducing agent.

5. The colloid solution of claim 1, wherein ethyl alcohol is a biocompatible reducing agent.

6. The colloid solution of claim 1, wherein glucose is a biocompatible reducing agent.

7. The colloid solution of claim 1, wherein tetraethylammonium hydroxide or tetraisopropylammonium hydroxide or tetrabutylammonium hydroxide or tetrapentylammonium hydroxide is tetraalkylammonium hydroxide.

8. The colloid solution of claim 1, wherein sodium tetrachloroaurate (III) is a gold salt.

9. The colloid solution of claim 1, wherein the average size of gold [Au⁰] nanoparticles is within 5-6 nm.

10. A method of obtaining a colloidal solution of gold nanoparticles in a nonaqueous polar solvent, preferably, in dimethyl sulfoxide, of claim 1, characterized in that gold salt is reduced by a biocompatible reducing agent in an alkaline medium when the solution of a gold salt and dimethyl sulfoxide interacts with a biocompatible reductant, which requires an alkaline medium to reduce gold ions to gold nanoparticles [Au⁰], dimethyl sulfoxide and tetraalkylammonium hydroxide, and the resulting colloidal solution is then adjusted to neutral pH.

11. The method of claim 10, wherein the resulting colloidal solution is adjusted to neutral pH by adding an organic acid to the resulting colloidal solution.

12. The method of claim 10, wherein ascorbic acid is used as a biocompatible reducing agent.

13. The method of claim 10, wherein glycerine is used as a biocompatible reducing agent.

14. The method of claim 10, wherein hydrogen peroxide is used as a biocompatible reducing agent.

15. The method of claim 10, wherein ethyl alcohol is used as a biocompatible reducing agent.

16. The method of claim 10, wherein glucose is used as a biocompatible reducing agent.

17. The method of claim 10, wherein tetraethylammonium hydroxide or tetraisopropylammonium hydroxide or tetrabutylammonium hydroxide or tetrapentylammonium hydroxide is used as tetraalkylammonium hydroxide.

18. The method of claim 10, wherein sodium tetrachloroaurate (III) is used as a gold salt.
